(19) United States
(12) Patent Application Publication
(10) Pub. No.: US 2009/0299067 A1
(43) Pub. Date: Dec. 3, 2009

(54) New synthesis of 2,6-diamino-3,5-dinitropyrazine-1-oxide from 2,6-diaminopyrazine-1-oxide

(76) Inventors: Philip F. Pagoria, Livermore, CA (US); Alexander R. Mitchell, Livermore, CA (US); Kason Bala, Winchester (GB)

Correspondence Address: Lawrence Livermore National Security, LLC, LAWRENCE LIVERMORE NATIONAL LABORATORY, PO BOX 808, L-703, LIVERMORE, CA, 94551-0808, US

(21) Appl. No.: 12/130,444

(22) Filed: May 30, 2008

(51) Int. Cl. C07D 241/20

(52) U.S. Cl. 544/408

(57) ABSTRACT

A method for the synthesis of 2,6-Diamino-3,5-dinitropyrazine-1-oxide including nitration of 2,6-diaminopyrazine-1-oxide. The step of nitration may use nitrating agents 20% Oleum/100% HNO3, 100% HNO3 and conc. H2504/100% HNO3 or nitronium tetrfluoraoborate. The synthesis of 2,6-diaminopyrazine-1-oxide may involve both the oxidation of 2,6-diaminopyrazine, the amination of a precursor 2,6-disubstitutedpyrazine-1-oxide or direct formation of the 2,6-diaminopyrazine-l-oxide from the precursor, 2-amino-6-hydroxamino-pyrazine-1-oxide.

The United States Government has rights in this invention pursuant to Contract No. DE-AC52-07NA27344 between the United States Department of Energy and Lawrence Livermore National Security, LLC for the operation of Lawrence Livermore National Laboratory.

### BACKGROUND

1. Field of Endeavor

The present invention relates to synthesis of 2,6-Diamino-3,5-dinitropyrazine-1-oxide (LLM-105) and more particularly to synthesis of 2,6-diamino-3,5-dinitropyrazine-1-oxide (LLM-105) from 2,6-diaminopyrazine-1-oxide.

2. State of Technology

The article, “Synthesis, Scale-up and Experimental Testing of LLM-105 (2,6-Diamino-3,5-dinitropyrazine-1-oxide;” by Philip Pagoria, Alexander Mitchell, Robert Schmidt, Randall Simpson, Frank Garcia, Jerry Forbes, Jack Cutting, Ronald Lee, Rosalind Swansiger, and D. Mark Hoffman; in Insensitive Munitions and Energetic Materials Technology Symposium, Meeting No. 956; National Defense Industrial Association: San Diego, Calif., 1998; provides the following state of technology information. “From this latter effort we have synthesized, 6-diamino-3,5-dinitropyrazine-1-oxide (LLM-105), an insensitive energetic material with 25% more energy than TATS (81% of HMX). The energy content and thermal stability of this material make it very interesting for several applications, including insensitive boosters and detonators, and deep oil well perforation.”

The article, “Heterocyclic amines and Aminesamines and Amines. Part XVL. “2,6-Diaminopyrazine and its I Oxide from iminodiacetonitrile;” by Nandkishar R. Barot and John A. Elvidge; in A. J. Chem. Soc. Perkin Trans. I 1973, 606-12; provides the following state of technology information. “With palladium-charcoal, 2,6-bishydroxyimiinopiperazine (made from iminodiacetonitrile and hydroxylamine at 70°) underwent hydrogen transfer to yield 2,6-diaminopyrazine, characterised as the diacetyl derivative.”

The article, “X-ray structural study of three derivatives of dinitropyrazine,” by B. B. Averkiev, M. Yu. Antipin, I. L. Yudin, and A. B. Sheremetev in the Journal of Molecular Structure, Volume 606, Issues 1-3, 27 Mar. 2002, Pages 139-146 provides the following state of technology information: “In recent years, a series of more powerful explosives and novel generation of energetic materials has been reported. In particular, azetidine-based explosives such as 1,3,3-trinitroazetidine (TNAZ) or CL-20), carbopolycyclic cage-compounds such as octanitrocubane (ONC), nitrofurazans such as 3-(4-nitrofurazan-3-NNO-azoxy)-4-(4-nitrofurazan-3-ONN-azoxy)-furazan (DIOD/J), and nitrofuroxans such as 4,4-dinitroazofuroxan (DNAFO) were found to be even more dense and high-energy materials possessing excellent properties for applications . . . . The search for other new powerful insensitive energetic materials continues.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a method of synthesis of 2,6-diaminopyrazine-1-oxide (DAPO) from 6-amino-2-hydroximinopyrazine-1-oxide (AHAPO). The method includes reduction with catalytic transfer hydrogenation. One embodiment utilizes palladium hydroxide as a catalyst and utilizing a hydrogen source of ammonium formate or formic acid. One embodiment utilizes palladium hydroxide on carbon catalyst. In one embodiment the step of catalytic transfer hydrogenation utilizes a metal catalyst of platinum, rhodium, ruthenium or palladium and utilizing a catalyst support of carbon, charcoal, alumina, barium carbonate, calcium carbonate, or barium sulfate.

The present invention provides a method for the synthesis of 2,6-Diamino-3,5-dinitropyrazine-1-oxide (LLM-105) including nitration of 2,6-diaminopyrazine-1-oxide. In one embodiment the step of nitration uses nitrating agents 20% Oleum/100% HNO₃, 100% HNO₃ and conc. H₂SO₄/100% HNO₃ or nitronium tetrfluoraoborate. One embodiment includes both the oxidation of 2,6-diaminopyrazine and the amination of a precursor 2,6-disubstitutedpyrazine-1-oxide or direct formation of the 2,6-diaminopyrazine-1-oxide (DAPO) from the precursor, 2-amino-6-hydroxamino-pyrazine-1-oxide (AHAPO).

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

The present invention provides synthesis of 2,6-Diamino-3,5-dinitropyrazine-1-oxide (LLM-105) based on the nitration of 2,6-diaminopyrazine-1-oxide with various nitrating agents including 20% Oleum/100% HNO₃, 100% HNO₃ and conc. H₂SO₄/100% HNO₃ or nitronium tetrfluoraoborate. The synthesis of 2,6-diaminopyrazine-1-oxide may involve both the oxidation of 2,6-diaminopyrazine, the amination of a precursor 2,6-disubstitutedpyrazine-1-oxide or direct formation of the 2,6-diaminopyrazine-1-oxide (DAPO) from the precursor, 2-amino-6-hydroxamino-pyrazine-1-oxide (AHAPO).

Background—2,6-Diamino-3,5-dinitropyrazine-1-oxide (LLM-105) was discovered at the Lawrence Livermore National Laboratory in 1994. LLM-105 is a thermally stable, insensitive molecule with a crystal density of 1.918 g/cc and a decomposition point of >350° C.

Applicants investigated whether the nitration of DAPO was possible since the nitration of 2,6-diaminopyrazine was shown to give only decomposition products. Applicants were encouraged by reports that heterocyclic amine-N-oxides are more aromatic than their heterocyclic amine precursors and are more stable to electrophilic addition. The precursor to DAPO, 2-amino-6-hydroxaminopyrazine-1-oxide (AHAPO) had been synthesized earlier from iminodiacetonitrile. Applicants reduced AHAPO to DAPO using a procedure which involved dissolving AHAPO in AcOH and treating it with H₂ at 45 psi at room temperature for 1 h in the presence of Adam's catalyst (PtO₂). DAPO is a stable white solid with a melting point of 294-5° C. It was demonstrated that DAPO could be nitrated with 100% HNO₃ in 20% oleum to give pure LLM-105 (by IR and DSC analysis), albeit in low yield. This experiment confirmed the viability of this approach.

This approach has three-fold attractiveness. First, an energetic compound is not produced until the final step, resulting in a synthesis that would be both safer and easier to scale-up. Secondly, the N-oxide is formed prior to nitration so the current problem of incomplete oxidation of the ANPZ to LLM-105 would be eliminated. Finally, the starting material, iminodiacetonitrile is inexpensive and readily available.

There are drawbacks to this synthesis. AHAPO is produced in low yields from diiminoacetonitrile and a significant amount of the main product (2,6-bis(hydroximino)piperazine-hydroxylamine hydrochloride complex) is formed that must be removed by filtration. Another drawback is that the reported reduction of AHAPO to DAPO requires the use of the flammable gas, hydrogen, under pressure for the conversion.

To address the drawback of using of hydrogen in the reduction step, transfer hydrogenation was investigated as a safe alternative for the reduction of AHAPO. Transfer hydrogenation requires a catalyst (such as Pearlmann's catalyst (20% Pd/C)) but utilizes either formic acid or ammonium formate (AF) as the hydrogen source, thus allowing the reduction to be performed at ambient pressure. When a transfer hydrogenation was performed on AHAPO with Pearlmann's catalyst using formic acid as both the solvent and the hydrogen source the reduction was somewhat successful, in that, by TLC analysis after 3 min., DAPO was already produced. Unfortunately, when the reaction mixture was allowed to stir for 2 h at room temperature the reduction yielded a complex set of products that did not include DAPO. It is believed the reduction went past DAPO, forming 2,6-diaminopyrazine, which was formylated by the excess formic acid present. Applicants believe by careful control of the amount catalyst, resident time, and possibly diluting the mixture with a co-solvent such as MeOH, this procedure may be optimized to produce DAPO in good yields and purity.

Several small-scale experiments (15-20 mg) were performed that used transfer hydrogenation with either formic acid or ammonium formate as the hydrogen source in MeOH in the presence of Pearlmann's catalyst, to test whether introduction of a co-solvent such as MeOH would allow some control over the extent of the reduction. In fact MeOH retarded the reduction to such an extent that formic acid was not effective as a hydrogen source for the reduction of AHAPO under the chosen conditions. Ammonium formate was effective but the reduction required an excess of both the catalyst and ammonium formate and the conversion of AHAPO to DAPO took 18 h (monitored by TLC analysis).

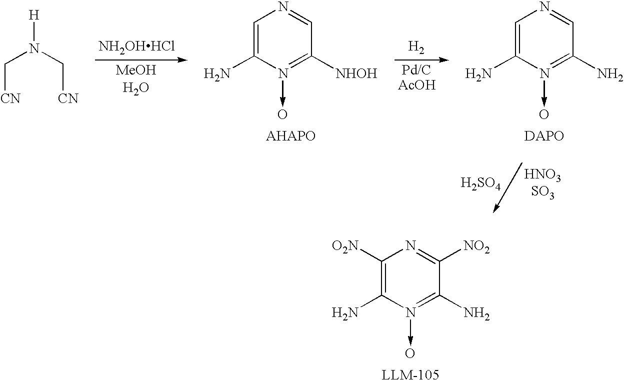

### EXPERIMENTAL

2,6-diamino-3,5-dinitropyrazine-1-oxide: Into a 10 mL 2-necked pear-shaped flask equipped with a drying tube, thermometer and stir bar was placed 20% oleum (1 mL). With stirring and cooling from an ice-acetone bath, 100% HNO₃ was added dropwise at <10° C. To this nitrating mixture 2,6-diaminopyrazine-1-oxide (DAPO) (150 mg, 1.2 mmol) was added portion-wise at <5° C. The DAPO was insoluble in the nitrating mixture and became a gummy solid that slowly congealed together. The mixture was stirred <5° C. 4 h, the DAPO slowly dissolving after about 1.5 h to yield a clear yellow solution. An aliquot was taken after stirring at <5° C. for 1.25 h and quenched into water (10 mL). Upon sitting 1 h a yellow-orange precipitate formed that was collected by suction filtration and washed with water to yield yellow-orange needles. An IR spectrum of this material suggests it is predominantly the mono-nitrated product with a small amount of LLM-105. Therefore the remaining nitrating mixture was allowed to warm to room temperature and stir 2 h. The reaction mixture was poured into water (20 mL) to yield a yellow solid that was collected by suction filtration, washed with water (2×10 ml) and allowed to dry at room temp. overnight under suction. The yellow solid was dried further at 70° C. under vacuum to yield 25 mg of a yellow solid. An IR spectrum showed it to be essentially pure LLM-105.

Transfer Hydrogenation of 2-amino-6-hydroxyaminopyrazine-1-oxide: Into a screw cap test tube equipped with a stir bar was placed sequentially 2-amino-6-hydroxyaminopyrazine-1-oxide (20 mg), MeOH (3 mL), 20% Pd/C (55 mg) and ammonium formate (50 mg) and the mixture was stirred vigorously at room temp. Aliquots were taken at 1 h, 5 h and 18 h intervals and investigated by TLC analysis (Silica gel, Ethyl acetate, DMF, conc, NH₄OH 16:3:1) using the color reagent 5% FeCl₃—H₂O in MeOH. After 1 h there was very little conversion, at 5 h there was approximately 50% conversion and at 18 h there was complete conversion.

The present invention provides a method of synthesis of 2,6-diaminopyrazine-1-oxide (DAPO) from 6-amino-2-hydroximinopyrazine-1-oxide (AHAPO). The method includes reduction with catalytic transfer hydrogenation. One embodiment utilizes palladium hydroxide as a catalyst and utilizing a hydrogen source of ammonium formate or formic acid. One embodiment utilizes palladium hydroxide on carbon catalyst. In one embodiment the step of catalytic transfer hydrogenation utilizes a metal catalyst of platinum, rhodium, ruthenium or palladium and utilizing a catalyst support of carbon, charcoal, alumina, barium carbonate, calcium carbonate, or barium sulfate.

The present invention provides a method for the synthesis of 2,6-Diamino-3,5-dinitropyrazine-1-oxide (LLM-105) including nitration of 2,6-diaminopyrazine-1-oxide. In one embodiment the step of nitration uses nitrating agents 20% Oleum/100% HNO₃, 100% HNO₃ and conc. H₂SO₄/100% HNO₃ or nitronium tetrfluoraoborate. One embodiment includes both the oxidation of 2,6-diaminopyrazine and the amination of a precursor 2,6-disubstitutedpyrazine-1-oxide or direct formation of the 2,6-diaminopyrazine-1-oxide (DAPO) from the precursor, 2-amino-6-hydroxamino-pyrazine-1-oxide (AHAPO). In one embodiment the step of nitration of 2,6-diaminopyrazine-1-oxide utilizes a nitrating medium of 70-100% nitric acid and oleum (sulfuric acid containing 5-60% sulfur trioxide). In another embodiment the step of nitration of 2,6-diaminopyrazine-1-oxide utilizes a nitrating medium of 90-100% nitric acid and a strong acid. In one embodiment the strong acid is trifluoroacetic acid. In one embodiment the strong acid is methanesulfonic acid. In one embodiment the strong acid is trifluoromethanesulfonic acid.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. (canceled)

2. (canceled)

3. (canceled)

4. (canceled)

5. A method for the synthesis of 2,6-Diamino-3,5-dinitropyrazine-1-oxide comprising the step of:
nitration of 2,6-diaminopyrazine-1-oxide, said step of nitration of 2,6-diaminopyrazine-1-oxide using nitrating agents 20% oleum/100% HNO₃, concentrated H₂SO₄/100% HNO₃ or nitronium tetrafluoroborate.

6. (canceled)

7. (canceled)

8. (canceled)

9. (canceled)

10. (canceled)

11. (canceled)
